(19) United States
(12) Patent Application Publication
(10) Pub. No.: US 2013/0023573 A1
(43) Pub. Date: Jan. 24, 2013

(54) ALPHA-2 ADRENERGIC MODULATORS FOR TREATING VISUAL DISORDERS MEDIATED BY CENTRAL VISUAL PROJECTIONS FROM THE EYE

(71) Applicants: Ursula V. Staubli, Laguna Beach, CA (US); Alan C. Foster, Rancho Santa Fe, CA (US); Daniel W. Gil, Corona Del Mar, CA (US); John E. Donello, Dana Point, CA (US)

(72) Inventors: Ursula V. Staubli, Laguna Beach, CA (US); Alan C. Foster, Rancho Santa Fe, CA (US); Daniel W. Gil, Corona Del Mar, CA (US); John E. Donello, Dana Point, CA (US)

(21) Appl. No.: 13/552,217

(22) Filed: Jul. 18, 2012

Related U.S. Application Data
(60) Provisional application No. 61/510,521, filed on Jul. 22, 2011.

(51) Int. Cl. A61K 31/4184; A61P 27/02; A61K 31/4174

(52) U.S. Cl. 514/392; 514/400

(57) ABSTRACT

The present invention relates to a method for treating visual disorders mediated by lateral geniculate nucleus, superior colliculus and the visual cortex by administering to a patient in need of such treatment, compounds acting at the alpha 2 adrenergic receptors.

### CROSS-REFERENCE TO RELATED APPLICATIONS

This application claims the benefit of U.S. Provisional Application Ser. No. 61/510,521, filed on Jul. 22, 2011, which is incorporated by reference herein in its entirety.

### FIELD OF THE INVENTION

The present invention relates to a method for treating visual disorders mediated by lateral geniculate nucleus, superior colliculus and the visual cortex by administering to a patient in need of such treatment compounds acting at the alpha 2 adrenergic receptors.

### BACKGROUND OF THE INVENTION

The compound (5-bromo-quinoxalin-6-yl)-imidazolidin-2-ylidene-amine is generically known as brimonidine; its tartrate salt is sold under the trademark ALPHAGAN®P (available from Allergan, Inc.). Pharmacological activation of the alpha 2 adrenergic receptor by brimonidine is a well established treatment for various visual disorders of the eye. Alpha 2 adrenergic agonists also have physiological effects beyond the eye in the central nervous system where they interact with the adrenergic central pathways. Thus, alpha 2 adrenergic agonists might also be beneficial for treating visual system disorders mediated by central visual areas, including, but not limited to visual cortex.

The visual cortex is one synapse removed from the eye and integrates visual signals generated by the retina. It is thus essential for decoding, processing and transforming visual inputs originating in the eye, and proper visual cortical function is necessary for normal vision. Noradrenaline released from the nerve terminals in visual cortex gates experience dependent modification of visual responsiveness including ocular dominance shifts after monocular deprivation (Marrocco, R T et al. 1987).

The effect of select alpha 2 adrenergic agonists was investigated in the visual cortex using brain slices prepared from primary visual cortex to determine possible drug interactions with the visual cortex plasticity mechanisms, in particular long-term potentiation (LTP). LTP serves as a cellular model for visual cortex plasticity and has functional consequences on visual evoked responses (Cooke and Bear, 2010). These alpha 2 adrenergic agonists are: 4-bromo-N-(imidazolidin-2-ylidene)-1H-benzimidazol-5-amine and (S)-(3-(1-(1H-imidazol-4-yl)ethyl)-2-methylphenyl)methanol structures represented below:

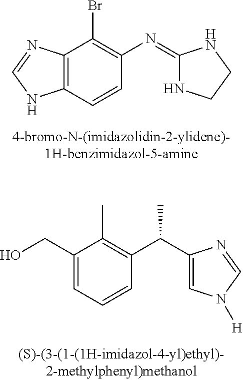 [SMILES: Brc1c(N=C2NCCN2)ccc2[nH]cnc12 ; Cc1c(CO)cccc1[C@H](C)c1c[nH]cn1]

Compound [3-(1-(1H-imidazol-4-yl)ethyl)-2-methylphenyl] methanol is known as a selective modulator of the alpha 2 adrenergic receptors. [3-(1-(1H-imidazol-4-yl)ethyl)-2-methylphenyl] methanol and its (S) enantiomer are described in Journal of Chromatography, (1997), 762(1+2), 281-291 by Hui, Y.-H et al.

[3-(1-(1H-imidazol-4-yl)ethyl)-2-methylphenyl]methanol is described in “Synthesis of detomidine and medetomidine metabolites: 1,2,3-trisubstituted arenes with 4′(5′)-imidazolylmethyl groups” in Journal of Heterocyclic Chemistry (1993), 30(6), (1645-1651) by Stoilov et al.

Kavanagh et al. describe [3-(1-(1H-imidazol-4-yl)ethyl)-2-methylphenyl]methanol in “Synthesis of Possible Metabolites of Medetomidine {1-(2,3-dimethylphenyl)-1-[imidazol-4(5)-yl]ethane” in Journal of Chemical Research, Synopses (1993), (4), 152-3. [3-(1-(1H-imidazol-4-yl)ethyl)-2-methylphenyl)methanol] is described by Salonen et al. in “Biotransformation of Medetomidine in the Rat” in Xenobiotica (1990), 20(5), 471-80.

PCT International Patent Application WO 2010093930 A1 discloses [3-(1-(1H-imidazol-4-yl)ethyl)-2-methylphenyl]methanol and its (S) and (R) enantiomers and their use for treating pain.

Compound 4-bromo-N-(imidazolidin-2-ylidene)-1H-benzimidazol-5-amine may be prepared according to the disclosure of U.S. Pat. No. 6,495,583 B1 which is hereby incorporated by reference in its entirety. Acheampong et al. have shown in Xenobiotica, February 2007, Vol. 37(2), pages 205-220 that this compound was found in trace amounts in the urine of rats after administration of an oral dose of brimonidine tartrate.

### SUMMARY OF THE INVENTION

It is an object of the invention to provide a method for treating visual disorders mediated by the visual cortex comprising administering to a patient in need of such treatment, a therapeutically effective amount of a pharmaceutical composition comprising an alpha 2 agonist and a pharmaceutically acceptable diluent or carrier

It is a further object of the invention to provide a method of treating visual disorders mediated by the visual cortex comprising administering to a patient in need of such treatment, a therapeutically effective amount of 4-bromo-N-(imidazolidin-2-ylidene)-1H-benzimidazol-5-amine or a pharmaceutically acceptable salt thereof.

It is a further object of the invention to provide a method of treating visual disorders mediated by the visual cortex comprising administering to a patient in need of such treatment, a therapeutically effective amount of (S)-(3-(1-(1H-imidazol-4-yl)ethyl)-2-methylphenyl)methanol or a pharmaceutically acceptable salt thereof.

### BRIEF DESCRIPTION OF THE DRAWINGS

FIG. 1 shows dose-dependent facilitation of LTP in rat visual cortex by 4-bromo-N-(imidazolidin-2-ylidene)-1H-benzimidazol-5-amine with a threshold dose of 3 nM.

FIG. 2 shows dose-dependent facilitation of LTP in rat visual cortex by (S)-(3-(1-(1H-imidazol-4-yl)ethyl)-2-methylphenyl)methanol with a threshold dose of 100 nM.

### DETAILED DESCRIPTION OF THE INVENTION

A prime example of a visual system disorder mediated by visual cortex is amblyopia. Amblyopia is defined as poor or indistinct vision by an eye that is physically normal. Amblyopia can be initiated by poor transmission of the visual image to the visual cortex during childhood. Abnormal visual processing may be caused by form deprivation (i.e. cataracts), anisomometropia (different retinal image size, or magnification, in each eye), or suppression resulting from strabismus (misalignment of the eyes). A prolonged transmission of poor quality visual images induces a physiological change within the visual cortex that alters the perception within the visual cortex. Briefly, the visual cortex will “ignore” the poor vision from one eye. Hence, amblyopia often lacks visual acuity and stereopsis. Amblyopia treatments include occlusion therapy with full-time or part-time patches, adhesive patches, opaque contact lenses, occluders mounted on spectacles, and adhesive tape on glasses or vision therapy with medication (such as atropine) or surgery for eye turn or cataract.

In addition to amblyopia, visual disorders mediated by visual cortex include, but are not limited to stroke-induced blindness, visual dysfunction in Parkinson's disease and Alzheimer's disease, seizure-induced cortical blindness, epileptic blindness, and induced visual dysfunction including but not limited and to multiple sclerosis (MS)-induced visual dysfunction, and congenital and childhood myotonic dystrophy type 1-induced visual dysfunction.

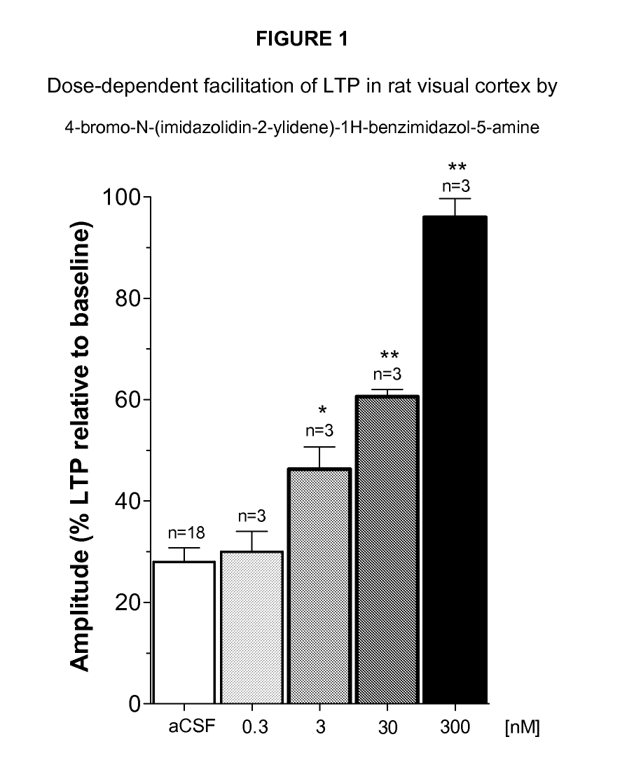

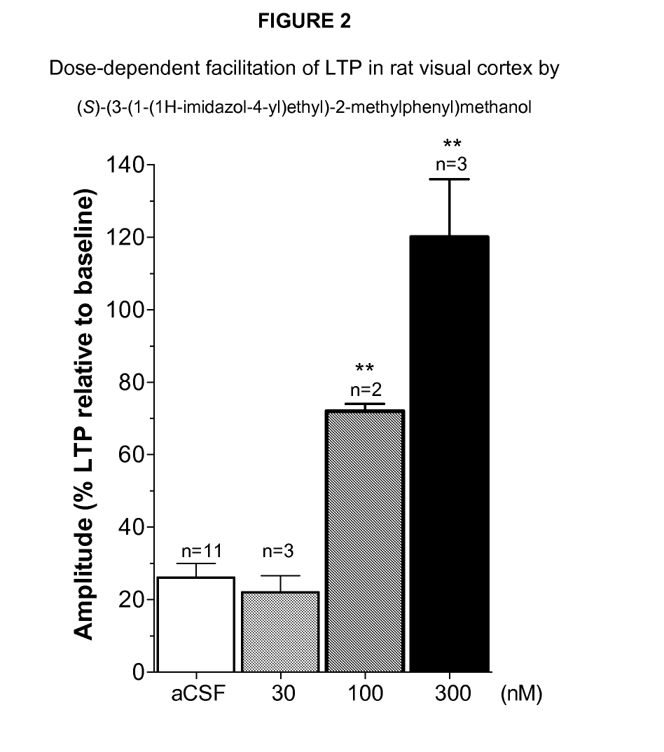

It has been discovered that compound 4-bromo-N-(imidazolidin-2-ylidene)-1H-benzimidazol-5-amine, administered to visual cortex slices produced a marked and dose-dependent enhancement of LTP, with a threshold dose of 3 nM (FIG. 1). A significant dose-dependent facilitatory activity was also found when testing another alpha 2 adrenergic agonist, (S)-(3-(1-(1H-imidazol-4-yl)ethyl)-2-methylphenyl)methanol, for LTP enhancement, with a threshold dose of 100 nM (FIG. 2). It has been determined that pharmacological activation of alpha 2 receptors has powerful facilitatory effects on LTP formation in the visual cortex. These findings are unexpected because there are reports that alpha 2 receptor activation may suppress LTP formation in brain areas such as the hippocampus and the amygdala (DeBock et al, 2003; Lim et al, 2010; Takamatsu et al, 2008), two subcortical sites that are however not essential for visual function.

The present results indicate that alpha 2 adrenergic agonists including, but not limited to 4-bromo-N-(imidazolidin-2-ylidene)-1H-benzimidazol-5-amine and (S)-(3-(1-(1H-imidazol-4-yl)ethyl)-2-methylphenyl)methanol, benefit visual disorders mediated by central cortical plasticity.

This invention provides a method for treating visual system disorders mediated by the visual cortex by administering to a patient in need of such treatment, 4-bromo-N-(imidazolidin-2-ylidene)-1H-benzimidazol-5-amine or (S)-(3-(1-(1H-imidazol-4-yl)ethyl)-2-methylphenyl)methanol or a pharmaceutically acceptable salt thereof.

To “treat,” as used here, means to deal with medically. It includes, for example, administering a compound of the invention to prevent the onset of a disorder, to alleviate its severity, and to prevent its reoccurrence.

It is a further object of the invention to provide a method for treating visual disorders mediated by the visual cortex visual disorder selected from: amblyopia, stroke-induced blindness, visual system disorder in Parkinson's disease and Alzheimer's disease, seizure-induced cortical blindness, epileptic blindness, multiple sclerosis (MS)-induced visual system disorder, and congenital and childhood myotonic dystrophy type 1-induced visual system disorder by administering to a patient in need of such treatment, a therapeutically effective amount 4-bromo-N-(imidazolidin-2-ylidene)-1H-benzimidazol-5-amine or a pharmaceutically acceptable salt thereof.

It is a further object of the invention to provide a method of treating visual disorders mediated by the visual cortex visual disorder selected from amblyopia, stroke-induced blindness, visual system disorder in Parkinson's disease and Alzheimer's disease, seizure-induced cortical blindness, epileptic blindness, multiple sclerosis (MS)-induced visual system disorder, and congenital and childhood myotonic dystrophy type 1-induced visual system disorder by administering to a patient in need of such treatment, a therapeutically effective amount of (S)-(3-(1-(1H-imidazol-4-yl)ethyl)-2-methylphenyl)methanol or a pharmaceutically acceptable salt thereof.

It is a further object of the invention to provide a method for treating amblyopia, comprising administering to a patient in need of such treatment, a therapeutically effective amount of 4-bromo-N-(imidazolidin-2-ylidene)-1H-benzimidazol-5-amine or a pharmaceutically acceptable salt thereof.

It is a further object of the invention to provide a method for treating amblyopia comprising administering to a patient in need of such treatment, a therapeutically effective amount of (S)-(3-(1-(1H-imidazol-4-yl)ethyl)-2-methylphenyl)methanol or a pharmaceutically acceptable salt thereof.

It is a further object of the invention to provide an article of manufacture comprising packaging material and a pharmaceutical agent contained within said packaging material, wherein the pharmaceutical agent is therapeutically effective for treating amblyopia and wherein the packaging material comprises a label which indicates the pharmaceutical agent can be used for amblyopia and wherein said pharmaceutical agent comprises an effective amount of 4-bromo-N-(imidazolidin-2-ylidene)-1H-benzimidazol-5-amine.

It is a further object of the invention to provide an article of manufacture comprising packaging material and a pharmaceutical agent contained within said packaging material, wherein the pharmaceutical agent is therapeutically effective for treating amblyopia and wherein the packaging material comprises a label which indicates the pharmaceutical agent can be used for amblyopia and wherein said pharmaceutical agent comprises an effective amount of (S)-(3-(1-(1H-imidazol-4-yl)ethyl)-2-methylphenyl)methanol.

The term “pharmaceutically acceptable salts” according to the invention include therapeutically active, non-toxic base or acid salt forms, which compound 4-bromo-N-imidazolidin-2-ylidene-1-H-benzimidazol-5-amine or (S)-(3-(1-(1H-imidazol-4-yl)ethyl)-2-methylphenyl)methanol are able to form.

The acid addition salt form of 4-bromo-N-(imidazolidin-2-ylidene)-1H-benzimidazol-5-amine or of (S)-(3-(1-(1H-imidazol-4-yl)ethyl)-2-methylphenyl)methanol that occur in the free form as a base, can be obtained by treating the free base with an appropriate acid such as an inorganic acid, for example hydrochloric acid, hydrobromic acid, sulfuric acid, phosphoric acid, nitric acid and the like; or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric, methylsulfonic, ethanesulfonic, benzenesulfonic, formic and the like (Handbook of Pharmaceutical Salts, P. Heinrich Stahal & Camille G. Wermuth (Eds), Verlag Helvetica Chemica Acta-Zürich, 2002, 329-345).

The activation of alpha 2 adrenergic receptors by 4-bromo-N-(imidazolidin-2-ylidene)-1H-benzimidazol-5-amine or (S)-(3-(1-(1H-imidazol-4-yl)ethyl)-2-methylphenyl)methanol confirms that alpha 2 adrenergic receptors are effective at enhancing cortical synaptic plasticity, and have therapeutic benefits in disorders where central visual plasticity needs to be restored or increased.

Alpha 2 adrenergic agonists may be administered at pharmaceutically effective amounts. Such amounts are normally the minimum dose necessary to achieve the desired therapeutic effect. The actual amount of the compound to be administered in any given case will be determined by a physician taking into account the relevant circumstances. In one embodiment, the compounds of the invention are administered at doses that are pharmaceutically effective but do not cause sedation. The patient may be given the compounds of the invention orally or by local delivery to the eye. Local delivery includes topical delivery, in which an ophthalmological acceptable formulation is instilled in the eye via an eye dropper or other applicator, delivery by injection into the eye.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

### General Procedure Followed in Obtaining Experimental Data

### Long-Term Potentiation in Visual Cortex Slice

Following decapitation of the anesthetized rat, the brain was rapidly removed and immersed in ice-cold artificial cerebrospinal fluid (ACSF) containing (in mM) NaCl 124, KCl 3, KH₂PO₄ 1.25, CaCl₂ 3.4, MgSO₄ 2.5, NaHCO₃ 26, and D-glucose 10. A block of visual cortex was created by removing the frontal ⅔ portion of the brain and the cerebellum. Coronal visual cortex slices of 350 μm thick were prepared from young adult (200-300 g) male Sprague-Dawley rats using a vibratome (VT 1000 S; Leica). The slices were maintained in an interface recording chamber perfused with preheated ACSF. Slices were continuously perfused with this solution at a rate of 1.00-1.50 ml/min while the surface of the slices was exposed to warm, humidified 95%O₂15% CO₂ and maintained at 31±1° C. Visual cortex slices were allowed to recover for 1 hr before recording began. A single stimulating and recording electrode was placed in layer IV and III, respectively, to generate and record field excitatory postsynaptic potentials (fEPSPs). Pulses were administered at 0.05 Hz using a current that produced a fEPSP that is 50% of the maximum spike free response. An input-output (IO) curve was done to determine the stimulation needed to achieve a stable baseline. Following a 15 min stable baseline recording period, a train of 5 theta bursts (each burst containing four pulses at 100 Hz with an inter-burst interval of 200 ms) was delivered to the slice. This was repeated 2 additional times with a 1 minute inter-train interval, and the level of LTP was recorded for at least 30 min. Changes in amplitude of the synaptic response were used to measure the extent of LTP, since the amplitude was determined to be the more consistent parameter than the slope of the response. Control LTP values were obtained from slices not treated with drug. Different slices were used to study drug effects on LTP. Compound 4-bromo-N-(imidazolidin-2-ylidene)-1H-benzimidazol-5-amine or (S)-(3-(1-(1H-imidazol-4-yl)ethyl)-2-methylphenyl)methanol) was infused after 15 min baseline recording for a duration of 20 minutes followed by LTP induction. Drug washout began 5 minutes after tetanization. Recording of the amplitude before, during, and after drug infusion was continuously done at 0.05 Hz. Statistical comparisons were done with LTP values recorded at 30 minutes after induction for drug-treated vs control slices (non-paired t-test).

In brief, brain slices from primary visual cortex were prepared and recording of evoked field responses was done as described in the ‘general procedure’ section. A typical LTP run began with establishing a stable baseline, then treatment for 20 min with 4-bromo-N-(imidazolidin-2-ylidene)-1H-benzimidazol-5-amine followed by LTP induction via brief high-frequency theta burst stimulation (TBS) and drug washout 5 min after TBS, and ended after monitoring the amount of LTP for at least 30 min. Control LTP was measured in a group of separate slices within the same chamber infused with aCSF. The amount of LTP present at 30 min after induction was used to compare drug effects at different concentrations relative to the control group. We found that LTP obtained in presence of different concentrations of alpha 2 agonist, 4-bromo-N-(imidazolidin-2-ylidene)-1H-benzimidazol-5-amine was significantly facilitated at 3, 30 and 300 nM compared to control slices. The percent increase in LTP was shown relative to the pre-LTP baseline for each condition. The results were reported in FIG. 1.

We found that LTP obtained in presence of different concentrations of alpha 2 agonist, (S)-(3-(1-(1H-imidazol-4-yl)ethyl)-2-methylphenyl)methanol was significantly facilitated at 100 and 300 nM compared to control slices. The percent increase in LTP was shown relative to the pre-LTP baseline for each condition. The results were reported in FIG. 2.

What is claimed is:

1. A method for treating a visual disorder mediated by the visual cortex comprising administering to a patient in need of such treatment, a therapeutically effective amount of a pharmaceutical composition comprising an alpha 2 adrenergic agonist and a pharmaceutically acceptable diluent or carrier.

2. The method of claim 1, wherein the alpha 2 adrenergic agonist is 4-bromo-N-(imidazolidin-2-ylidene)-1H-benzimidazol-5-amine or a salt thereof.

3. The method of claim 1, wherein the alpha 2 adrenergic agonist is (S)-(3-(1-(1H-imidazol-4-yl)ethyl)-2-methylphenyl)methanol or a salt thereof.

4. The method of claim 1, wherein the visual disorder comprises amblyopia, stroke-induced blindness, visual system disorder in Parkinson's disease and Alzheimer's disease, seizure-induced cortical blindness, epileptic blindness, multiple sclerosis induced visual system disorder, congenital and childhood myotonic dystrophy type 1-induced visual system disorder.

5. The method of claim 2, wherein the visual disorder is amblyopia.

6.

7. The method of claim 2 wherein the visual disorder is stroke-induced blindness.

8. The method of claim 2, wherein the visual disorder is in Parkinson's disease.

9. The method of claim 2, wherein the pharmaceutical composition is administered topically.

10. The method of claim 3, wherein the visual disorder is amblyopia.

11. The method of claim 3 wherein the visual disorder is stroke-induced blindness.

12. The method of claim 3, wherein the visual disorder is in Parkinson's disease.

13. The method of claim 3, wherein the pharmaceutical composition is administered topically.

14. An article of manufacture comprising packaging material and a pharmaceutical agent contained within said packaging material, wherein the pharmaceutical agent is therapeutically effective for amblyopia and wherein the packaging material comprises a label which indicates the pharmaceutical agent can be used for amblyopia and wherein said pharmaceutical agent comprises a therapeutically effective amount of 4-bromo-N-(imidazolidin-2-ylidene)-1H-benzimidazol-5-amine.

15. An article of manufacture comprising packaging material and a pharmaceutical agent contained within said packaging material, wherein the pharmaceutical agent is therapeutically effective for amblyopia and wherein the packaging material comprises a label which indicates the pharmaceutical agent can be used for amblyopia and wherein said pharmaceutical agent comprises a therapeutically effective amount of (S)-(3-(1-(1H-imidazol-4-yl)ethyl)-2-methylphenyl)methanol.
